(19) United States
(12) Patent Application Publication
(10) Pub. No.: US 2024/0371521 A1
(43) Pub. Date: Nov. 7, 2024

(54) FEDERATED IMITATION LEARNING FOR MEDICAL DECISION MAKING

(71) Applicant: NEC Laboratories America, Inc., Princeton, NJ (US)

(72) Inventors: Wenchao Yu, Plainsboro, NJ (US); Haifeng Chen, West Windsor, NJ (US); Wei Cheng, Princeton Junction, NJ (US)

(21) Appl. No.: 18/649,072

(22) Filed: Apr. 29, 2024

Related U.S. Application Data
(60) Provisional application No. 63/464,240, filed on May 5, 2023.

(51) Int. Cl. G16H 50/20; G06N 3/098

(52) U.S. Cl. CPC G16H 50/20; G06N 3/098

(57) ABSTRACT

Methods and systems for skill prediction include aggregating locally trained parameters from client systems to generate updated global parameters. Parameterized vectors from the client systems are clustered into prototype clusters. A centroid of each prototype cluster is determined and the parameterized vectors from the client systems are matched to centroids of the prototype clusters to identify sets of updated local prototype vectors. The updated global parameters and the updated local prototype vectors are distributed to the client systems.

### RELATED APPLICATION INFORMATION

This application claims priority to U.S. Patent Application No. 63/464,240, filed May 5, 2023, incorporated herein by reference in its entirety.

### BACKGROUND

### Technical Field

The present invention relates to federated learning and, more particularly, to federated imitation learning of medical treatment skills.

### Description of the Related Art

Imitation learning replicates experts' skills using a set of demonstrations. However, existing approaches to imitation learning are difficult to interpret, such that it is not a simple matter to understand why a trained imitation learning model has selected a particular action. Additionally, due to the scarcity of expert demonstrations from any given user, learning a policy from multiple different data silos introduces privacy challenges, particularly in privacy-sensitive fields such as healthcare.

### SUMMARY

A method for skill prediction includes aggregating locally trained parameters from client systems to generate updated global parameters. Parameterized vectors from the client systems are clustered into prototype clusters. A centroid of each prototype cluster is determined and the parameterized vectors from the client systems are matched to centroids of the prototype clusters to identify sets of updated local prototype vectors. The updated global parameters and the updated local prototype vectors are distributed to the plurality of client systems.

A system for skill prediction includes a hardware processor and a memory that stores a computer program. When executed by the hardware processor, the computer program causes the hardware processor to aggregate locally trained parameters from client systems to generate updated global parameters, to cluster parameterized vectors from the client systems into prototype clusters, to determine a centroid of each prototype cluster, to match the parameterized vectors from the client systems to centroids of the prototype cluster to identify sets of updated local prototype vectors, and to distribute the updated global parameters and the updated local prototype vectors to the client systems.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a federated imitation skill learning system that collects locally trained policy information from individual clients and that generates returns a global policy, in accordance with an embodiment of the present invention;

FIG. 2 is a block diagram of a skill prediction model that may be trained in a federated manner to provide interpretable action recommendations, in accordance with an embodiment of the present invention;

FIG. 3 is pseudo-code for federated imitation skill learning, in accordance with an embodiment of the present invention;

FIG. 4 is pseudo-code for local prototype projection, in accordance with an embodiment of the present invention;

FIG. 5 is a block/flow diagram of a method for federated imitation skill learning, in accordance with an embodiment of the present invention;

FIG. 6 is a block diagram of an exemplary healthcare facility where skill prediction is used to provide treatment recommendations and aid in medical decision making, in accordance with an embodiment of the present invention;

FIG. 7 is a block diagram of a computing device that can perform federated imitation skill learning, in accordance with an embodiment of the present invention;

FIG. 8 is a diagram of a neural network architecture that can be used as part of an imitation learning model, in accordance with an embodiment of the present invention; and

FIG. 9 is a diagram of a neural network architecture that can be used as part of an imitation learning model, in accordance with an embodiment of the present invention.

### DETAILED DESCRIPTION OF PREFERRED EMBODIMENTS

Federated skill learning may be used to learn a global policy from skill demonstrations collected from multiple locations. The federated skill learning further provides explainable interpretations to local users without violating privacy, data sovereignty, or relevant data control regulations. Examples may capture the varying patterns in the trajectories of expert demonstrations and may extract prototypical information as skills that provide implicit guidance for policy learning and explicit explanations in the reasoning process. Aggregation is coupled with the skill learning model to preserve global information utilization and to maintain local interpretability. Skills may be learned at the segment level, making them more flexible and transferable across different experts compared to trajectory-level formulations.

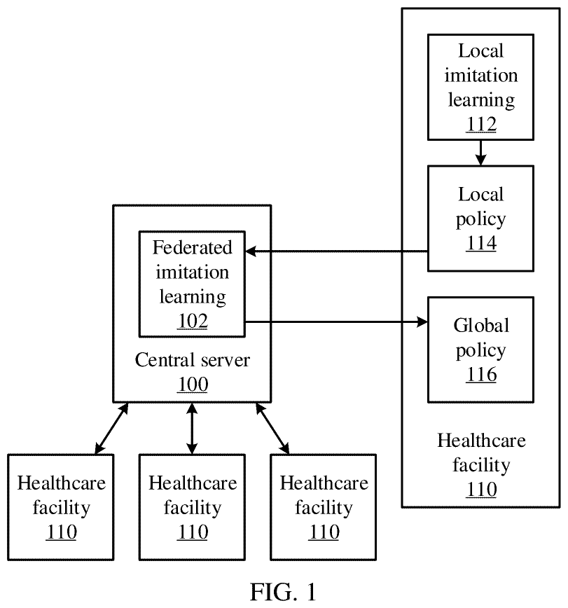

Referring now to FIG. 1, an illustration of federated imitation learning is shown. A central server 100 communicates with multiple clients 110. Clients 110 are described herein as healthcare facilities, but it should be understood that these clients 110 may be implemented as any type of system that performs independent skill learning as described herein. Each healthcare facility 110 performs local imitation learning using a set of locally derived expert demonstrations. For example, the treatment decisions by a given medical professional may be recorded, along with information regarding the patient's state, and may be used as a basis for imitation learning of a local policy 114. Information about the local policy 114, or the local policy itself, may be transmitted to the central server 100.

The central server 100 performs federated imitation learning 102 using the information from the healthcare facilities 110. The federated imitation learning 102 generates a global policy 116 which is transmitted back to the healthcare facilities, which use the global policy 116 to guide treatment decisions.

For example, a set of healthcare facilities 110 may all collaborate to cure a disease via imitation learning. Each hospital learns a model that discovers and explains the underlying skills used to treat the disease in a manner that preserves patient privacy. A contextual treatment policy is coupled with a learnable prototype set that includes parameterized vectors to represent prototypical symptoms and treatments after training. The skill may be formulated by combining multiple prototypes representing different treatment plans that correspond to different systems.

The local imitation learning 112 extracts the representations of segmented trajectories as candidates for skill learning. Parameterized vectors are optimized to have interpretation properties via multiple objects based on the candidates. On the server side, federated averaging may be used to aggregate the treatment policy networks and learnable prototype sets. However, treatment demonstrations can be heterogeneous across hospitals, leading to different disease treatment skills. To enable better knowledge sharing, clustering may be performed to identify he memberships of parameterized vectors from all healthcare facilities 110. As such, similar prototypical knowledge is aligned on the central server 100, which enhances local skill learning.

After training, each healthcare facility 110 has the global treatment policy 116 and a unique prototype set. Each parameterized vector is associated with a meaningful segment in the local training data, thereby identifying a prototype that is readily interpretable. Local data privacy is preserved, as the central server 100 does not access the data or its representation during the training process. Each healthcare facility can detect a new patient's symptoms and provide the underlying skill, with interpretations, by analyzing data that it is familiar with. The contextual treatment policy can recommend treatments, such as medications, based on a patient's status and an inferred skill.

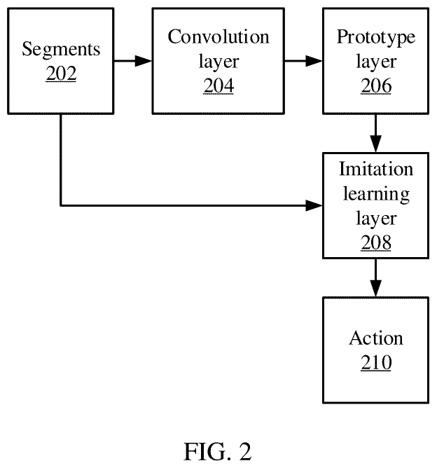

Referring now to FIG. 2, additional detail on Federated imitation learning 102 is shown. In federated imitation learning 102, a set of clients C aims to learn an interpretable skill learning model, for example including a machine learning model, including a client-specific prototype-layer 206 connecting the other learning components that generate segment representations 202 to establish the prototypes and construct skill embeddings.

Segment-level expert demonstrations 202 are used to perform imitation learning tasks. Each trajectory may therefore be divided into multiple non-overlapping segments of length m, resulting in a set of segments

$${\{ {[ ( {s}_{t}^{( i )} , {a}_{t}^{( i )} ) ]}_{t = 1}^{m} \}}_{i = 1}^{N} ,$$

where N is the number of segments from all trajectories and m is the number of time steps. Each state comes with the previous m−1 steps of states from the same trajectory, which encodes temporal dynamics up to the current step. Skill transitions across consecutive segments 202 are also captured in the model learning. Padding of the initial state in the input may be performed when needed.

A convolution layer 204 encodes temporal dynamics from the input segment and the prototype layer 206 generates skill embeddings. A contextual imitation learning layer 208 performs primitive actions 210 in response to the segments 202 and the prototypes.

Given an input segment at step t, [sₜ₋ₘ₊₁, . . . , sₜ₋₁, sₜ]∈ [symbol] ^{m×d}, where d denotes the feature dimension of each state, a representation rₜ may be extracted using two-dimensional convolution layer 204, which encodes the feature and temporal dynamics:

$${r}_{t} = Conv ( {s}_{t - m + 1 : t} ) = tanh ( {CAT}_{i = 0}^{h - 1} ( {W}_{i} ★ {s}_{t - m + 1 : {t}_{{b}_{i}}} ) )$$

where rₜ∈ [symbol] ^{h×1}, h is the number of kernels in convolution layer 204, Wᵢ∈ [symbol] ^{m×d} and bᵢ∈ [symbol] are the weight and bias terms for the i^{th} kernel, * is a two-dimensional cross-correlation operator, and CAT(⋅) is a concatenation function. A convolution-based encoder generates segment representations to efficiently extract salient information for segments with short lengths.

There is a set of k parameterized vectors P in the prototype layer 206, where P=[p₁, . . . , pₖ]∈ [symbol] ^{k×h}, with a dimensionality equal to rₜ. Each vector is optimized to be representative of and close to a set of similar segments in the representation space.

During a forward pass, the similarity between the segment representation rₜ and each parameterized vector may be evaluated by an exponential function based on their L₂ distance:

$${sim}_{{r}_{t} , {p}_{i}} = {e}^{- {‖ {r}_{t} - {p}_{i} ‖}_{2}^{2}} / {\sum }_{i = 1}^{k} {e}^{- {‖ {r}_{t} - {p}_{i} ‖}_{2}^{2}} .$$

All relative similarity scores may be concatenated to a similarity vector Wₛᵢₘ=[simᵣ_{ₜ}_{,p}_{₁}, simᵣ_{ₜ}_{,p}_{ᵢ} . . . , simᵣ_{ₜ}_{,p}_{ₖ}], which implies the importance of the corresponding vectors in the skill embedding generation. Thus, the skill embedding eₜ∈ [symbol] ^{1×h} is generated by the weighted combination of all parameterized vectors: eₜ=Wₛᵢₘ^{T}·P.

The segment representation rₜ may not be used directly in the final form of a skill embedding. Instead, the parameterized vectors may be used to reconstruct the skill information preserved by the segment representation, which renders a flexible interpretation structure. All parameterized vectors p are projected to prototypes that are segment representations of local training data, so that the underlying skill of a segment is explained by similar prototypes with high weights. This soft-skill combination can be rendered instead as a hard-skill selection by adopting a Gumbel softmax. However, the soft-skill combination tends to provide better flexibility and generalizability when encountering a new pattern in a complex task environment.

In the imitation learning layer 208, a contextual policy is built based on behavior cloning to learn a mapping from a state sₜ to an action aₜ in a supervised manner. The contextual policy is parameterized by θ and denoted as π_{θ}(aₜ|eₜ, sₜ), which takes the concatenation of skill embedding and the state as input. The skill embedding captures the varying patterns of expert trajectories and guides the agent to perform primitive actions more accurately:

aₜ←π_{θ}(aₜ|eₜ,sₜ)

The learning objectives for the imitation learning layer 208 include a segment-level imitation learning objective and multiple objectives that reinforce the interpretability of the final prototypes and skills in the non-overlapped segments. Each component is described herein based on a batch of segments in the training data:

$${\{ {[ ( {s}_{t}^{( i )} , {a}_{t}^{( i )} ) ]}_{t = 1}^{m} \}}_{i = 1}^{N} ,$$

with a batch size n and segment length m.

The first objective seeks to minimize the imitation learning loss:

$${ℒ}_{im} = {\sum }_{i = 1}^{n} {\sum }_{t = 1}^{m} {π}_{E} ( {a}_{t}^{( i )} , {s}_{t}^{( i )} ) log {π}_{θ} ( {a}_{t}^{( i )} \vert {e}_{t}^{( i )} , {s}_{t}^{( i )} )$$

where π_{E} denotes an expert policy that generates demonstrations.

A second objective regularizes the non-overlapped segment representation to be adjacent to its closest prototype, which enforces a clustering structure of segments in the representation space. For example, the smallest L₂ distance between non-overlapped rₜ₌ₘ⁽ⁱ⁾ and all parameterized vectors in P may be minimized:

$${ℒ}_{c} = {\sum }_{i = 1}^{n} {min}_{{p}_{j} ∈ P} {‖ {r}_{t = m}^{( i )} - {p}_{j} ‖}_{2}^{2}$$

A third objective reverse-regularizes each vector to be similar to a segment representation by minimizing the smallest L₂ distance between each pᵢ and a batch of non-overlapped segment representations

$${r}_{t = m}^{( j )} ∈ {ℛ}_{n} {=}^{def} {[ {[ Conv ( {s}_{t - m + 1 : t}^{( j )} ) ]}_{t = m} ]}_{j = 1}^{n} ,$$

which helps the downstream projection to evidence segments.

$${ℒ}_{e} = {\sum }_{i = 1}^{k} {min}_{{r}_{t = m}^{( j )} ∈ {ℛ}_{n}} {‖ {p}_{i} - {r}_{t = m}^{( j )} ‖}_{2}^{2}$$

The second and third objectives impose dual regularizations on the learning of the convolution layer 204 and the prototype layer 206 toward a clear representation structure for interpretation.

A fourth objective enforces a diverse structure of parameterized vectors to avoid redundancy and to improve the generalizability of resulting prototypes, where the L₂ distance between each pair of vector is penalized, with a threshold dₘᵢₙ:

$${ℒ}_{d} = {\sum }_{i = 1}^{k} {\sum }_{( j \neq i )}^{k} max ( 0 , {d}_{min} - {‖ {p}_{i} - {p}_{j} ‖}_{2}^{2}$$

As such, the full objective for learning client-specific prototypes may be written as:

$$ℒ = {ℒ}_{im} + {λ}_{1} {ℒ}_{c} + {λ}_{2} {ℒ}_{e} + {λ}_{3} {ℒ}_{d}$$

with non-negative weights λ₁, λ₂, and λ₃ being used to balance components toward an optimal solution.

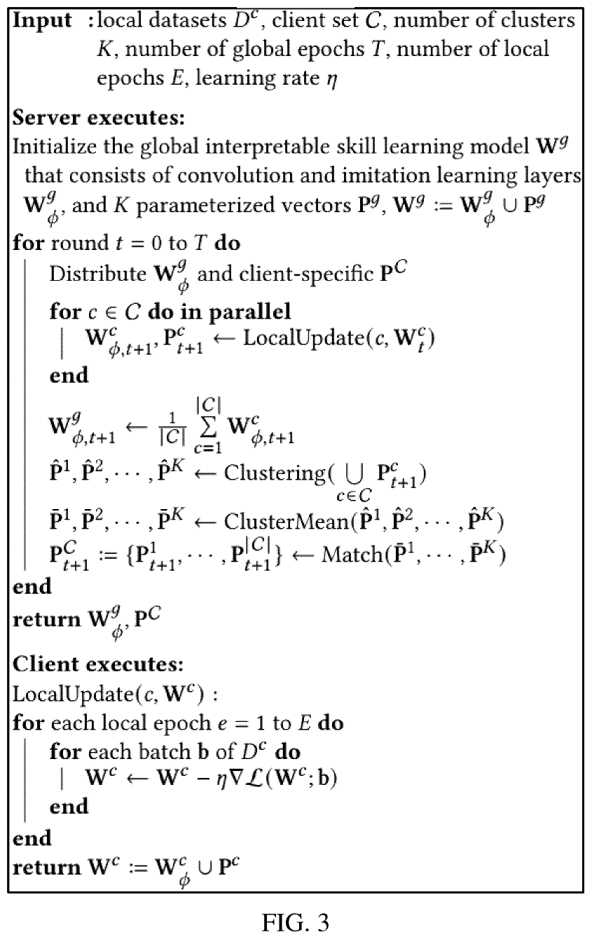

Referring now to FIG. 3, pseudo-code for training a federated skill model is shown. On the server-side, global knowledge alignment is conducted during the training stage. On the client-side, a local prototype projection is applied at model deployment, which yields interpretability without privacy concerns.

The central server 100 initializes and distributes the global policy 116 to local clients 110 at the beginning of federated imitation learning 102. The global policy 116 may be partitioned into two sets of parameters, one being having the parameterized vectors of the prototype layer 206, denoted as P^{g}, and the other having the weights of the convolution layer 204 and imitation learning layer 208, denoted as W^{g}.

For each global epoch, the server 100 aggregates local policies 114 from clients 110 after local imitation learning 112. The parameters W^{g} may be aggregated to generate segment representations and the final action, for example by averaging the values of the parameters across the different clients 110. However, parameterized vector sets can differ between clients 110 due to the heterogeneity of expert demonstrations across different clients. This discrepancy leads to a misalignment between local and global skills and thus aggregating P^{g} may yield sub-optimal prototypes for inference and reasoning.

Knowledge alignment may therefore be used by the central server 100. After receiving k×|C| parameterized vectors, the server 100 performs clustering, for example using K-means clustering and Gaussian mixture models with k clusters/components. The clustering identifies membership for each vector, and vectors with similar prototypical/skillful representations are aligned to same prototype clusters. After that, a centroid vector that represents a mode of skill is obtained by the mean of all vectors that belong to a given cluster. Each local vector may then be matched to the global centroid vector based on identified membership and may be distributed back to the clients 110. Each client then owns a specific vector set that best represents the prototypical information from its own data through the entire training process, where skillful knowledge is shared and aligned across different clients.

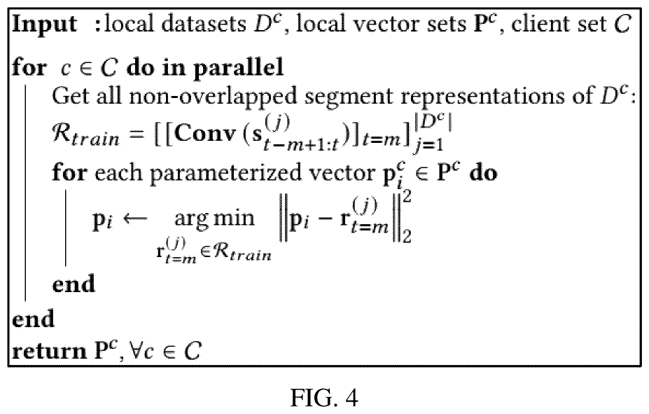

Referring now to FIG. 4, pseudo-code for local prototype projection is shown. Even if the training is completed and the local parameterized vector is well-regulated to be skillful, the returned local policy 114 is not readily interpretable. This is because the vectors are just approximations due to [symbol] and do not associate with actual data that represents explicit prototypes. To enable the interpretability of each client's local policy 114, local prototype projection is performed to each parameterized vector by assigning it to the training segment that has the smallest L₂ distance in the representation space. The vectors may then be translated to explicit prototypes that the local policy 114 can use to capture the varying patterns to construct a meaningful skill embedding. To preserve privacy, this step may be performed once for each client 110 during inference and reasoning.

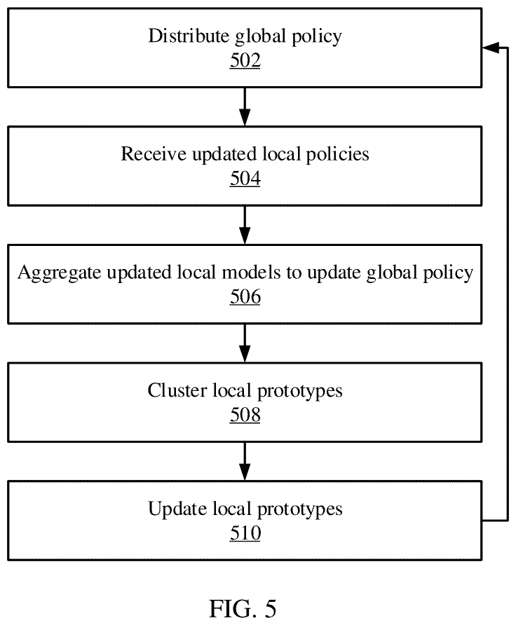

Referring now to FIG. 5, a server-side method for performing federated skill learning is shown. The server 100 distributes 502 a global policy 116 to the clients 110 by any appropriate wired or wireless communications medium and protocol. After the client 110 performs local imitation learning 112, the server 100 receives 504 the updated local policies 114 from the clients 110.

Block 506 aggregates the model parameters from the updated local policies 114 to update the global policy parameters. Block 508 furthermore clusters the local prototypes to generate centroid vectors, which block 510 uses to update the local prototypes. Processing then returns to block 502, where the updated global parameters and the updated local prototypes are distributed to the clients 110 as an updated global policy. This process may be repeated iteratively on a periodic basis or responsive to a triggering condition.

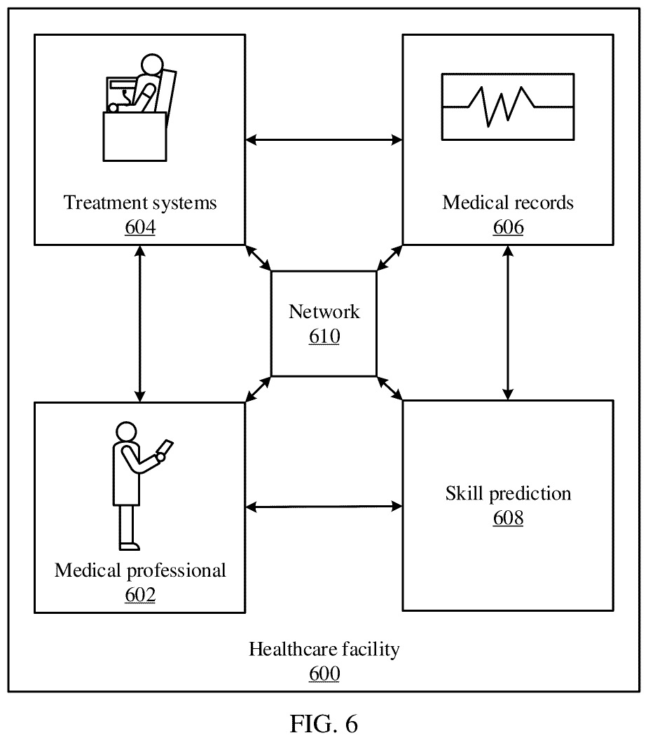

Referring now to FIG. 6, a diagram of skill prediction is shown in the context of a healthcare facility 600. Skill prediction 608 may be used to process patient information and to generate recommended actions based on the patient's present state. For example, information from patient history information and medical records 606 may be used to define a present state of the patient. Skill prediction 608 may use a trained policy to determine a skill or action to be implemented, and may further identify one or more local prototype vectors corresponding to the predicted skill so that the reasons for the selection of the skill may be interpreted.

The healthcare facility may include one or more medical professionals 602 who review information extracted from a patient's medical records 606 to determine their healthcare and treatment needs. These medical records 606 may include self-reported information from the patient, test results, and notes by healthcare personnel made to the patient's file. Treatment systems 604 may furthermore monitor patient status to generate medical records 606 and may be designed to automatically administer and adjust treatments as needed.

Based on the action selected by skill prediction 608, the medical professionals 602 may then make medical decisions about patient healthcare suited to the patient's needs. For example, the medical professionals 602 may select a treatment responsive to the patient's health condition that is based on the predicted skill, for example prescribing medications, surgeries, and/or therapies.

The different elements of the healthcare facility 600 may communicate with one another via a network 610, for example using any appropriate wired or wireless communications protocol and medium. Thus skill prediction 608 receives patient state information from medical professionals 602, from treatment systems 604, and from medical records 606. Skill prediction 608 may coordinate with treatment systems 604 in some cases to automatically administer or alter a treatment. For example, if the skill prediction 608 indicates that a particular treatment or medication should be administered or should be ceased, the treatment systems 604 may automatically administer or halt the treatment as appropriate.

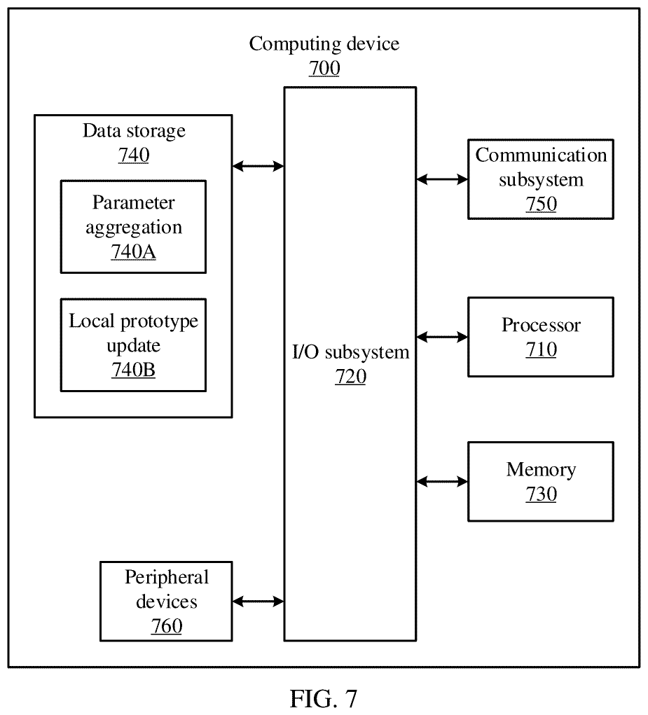

Referring now to FIG. 7, an exemplary computing device 700 is shown, in accordance with an embodiment of the present invention. The computing device 700 is configured to perform tissue analysis.

The computing device 700 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 700 may be embodied as one or more compute sleds, memory sleds, or other racks, sleds, computing chassis, or other components of a physically disaggregated computing device.

As shown in FIG. 7, the computing device 700 illustratively includes the processor 710, an input/output subsystem 720, a memory 730, a data storage device 740, and a communication subsystem 750, and/or other components and devices commonly found in a server or similar computing device. The computing device 700 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 730, or portions thereof, may be incorporated in the processor 710 in some embodiments.

The processor 710 may be embodied as any type of processor capable of performing the functions described herein. The processor 710 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 730 may be embodied as any type of volatile or non-volatile memory or data storage capable of performing the functions described herein. In operation, the memory 730 may store various data and software used during operation of the computing device 700, such as operating systems, applications, programs, libraries, and drivers. The memory 730 is communicatively coupled to the processor 710 via the I/O subsystem 720, which may be embodied as circuitry and/or components to facilitate input/output operations with the processor 710, the memory 730, and other components of the computing device 700. For example, the I/O subsystem 720 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 720 may form a portion of a system-on-a-chip (SOC) and be incorporated, along with the processor 710, the memory 730, and other components of the computing device 700, on a single integrated circuit chip.

The data storage device 740 may be embodied as any type of device or devices configured for short-term or long-term storage of data such as, for example, memory devices and circuits, memory cards, hard disk drives, solid state drives, or other data storage devices. The data storage device 740 can store program code 740A for parameter aggregation and 740B for updating local prototypes. Any or all of these program code blocks may be included in a given computing system. The communication subsystem 750 of the computing device 700 may be embodied as any network interface controller or other communication circuit, device, or collection thereof, capable of enabling communications between the computing device 700 and other remote devices over a network. The communication subsystem 750 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 700 may also include one or more peripheral devices 760. The peripheral devices 760 may include any number of additional input/output devices, interface devices, and/or other peripheral devices. For example, in some embodiments, the peripheral devices 760 may include a display, touch screen, graphics circuitry, keyboard, mouse, speaker system, microphone, network interface, and/or other input/output devices, interface devices, and/or peripheral devices.

Of course, the computing device 700 may also include other elements (not shown), as readily contemplated by one of skill in the art, as well as omit certain elements. For example, various other sensors, input devices, and/or output devices can be included in computing device 700, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 700 are readily contemplated by one of ordinary skill in the art given the teachings of the present invention provided herein.

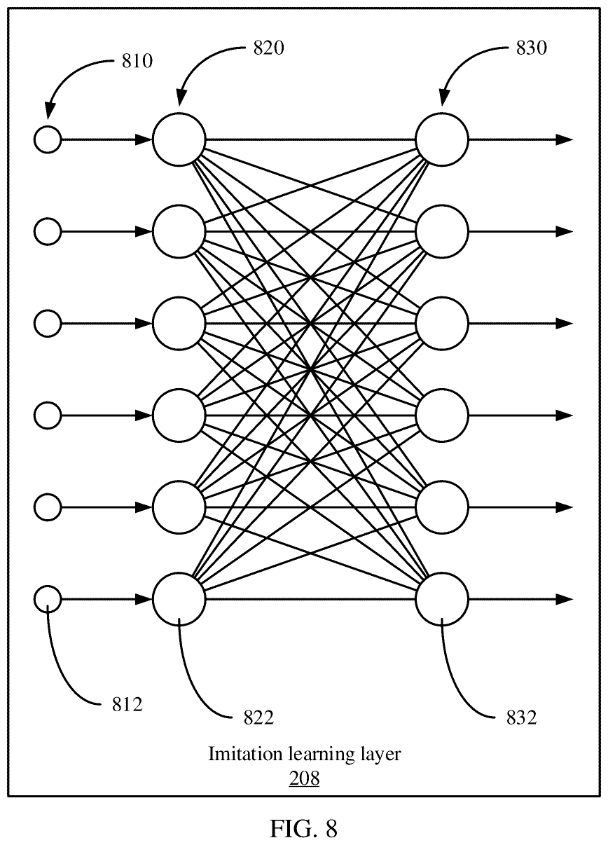

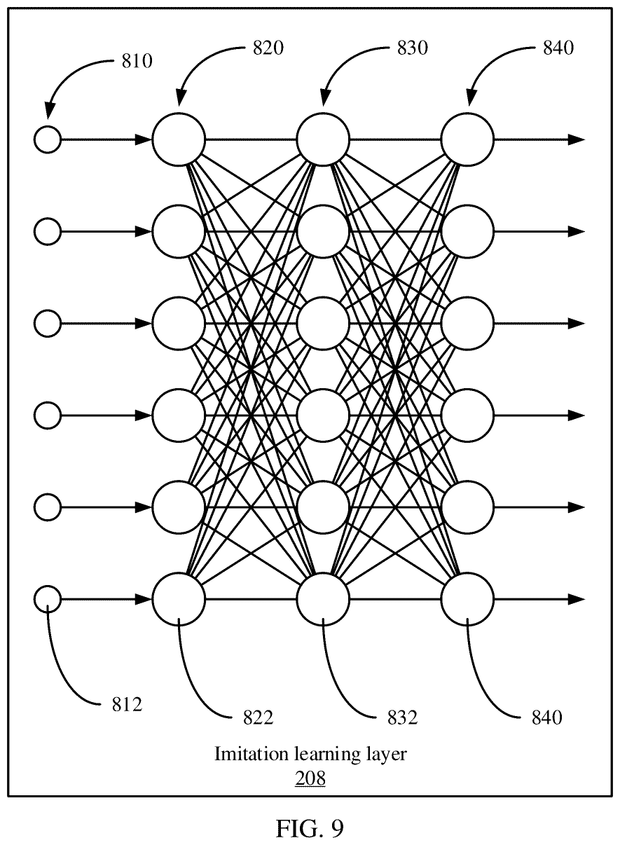

Referring now to FIGS. 8 and 9, exemplary neural network architectures are shown, which may be used to implement parts of the present models, such as the imitation learning layer 208. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 820 of source nodes 822, and a single computation layer 830 having one or more computation nodes 832 that also act as output nodes, where there is a single computation node 832 for each possible category into which the input example could be classified. An input layer 820 can have a number of source nodes 822 equal to the number of data values 812 in the input data 810. The data values 812 in the input data 810 can be represented as a column vector. Each computation node 832 in the computation layer 830 generates a linear combination of weighted values from the input data 810 fed into input nodes 820, and applies a non-linear activation function that is differentiable to the sum. The exemplary simple neural network can perform classification on linearly separable examples (e.g., patterns).

A deep neural network, such as a multilayer perceptron, can have an input layer 820 of source nodes 822, one or more computation layer(s) 830 having one or more computation nodes 832, and an output layer 840, where there is a single output node 842 for each possible category into which the input example could be classified. An input layer 820 can have a number of source nodes 822 equal to the number of data values 812 in the input data 810. The computation nodes 832 in the computation layer(s) 830 can also be referred to as hidden layers, because they are between the source nodes 822 and output node(s) 842 and are not directly observed. Each node 832, 842 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 832 in the one or more computation (hidden) layer(s) 830 perform a nonlinear transformation on the input data 812 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for skill prediction, comprising:
aggregating locally trained parameters from a plurality of client systems to generate updated global parameters;
clustering parameterized vectors from the client systems into prototype clusters;
determining a centroid of each prototype cluster;
matching the parameterized vectors from the client systems to centroids of the prototype clusters to identify sets of updated local prototype vectors; and
distributing the updated global parameters and the updated local prototype vectors to the plurality of client systems.

2. The method of claim 1, wherein aggregating the locally trained parameters includes averaging the locally trained parameters.

3. The method of claim 1, wherein determining the centroid of each prototype cluster includes identifying a mean of vectors in the prototype cluster.

4. The method of claim 1, wherein the locally trained parameters include parameters of a convolution layer and an imitation learning layer of a skill prediction model.

5. The method of claim 4, wherein the skill prediction model is implemented as a machine learning model.

6. The method of claim 4, wherein the skill prediction model accepts a patient's state as input and outputs a treatment recommendation.

7. The method of claim 6, wherein the treatment recommendation includes an interpretable identification of local prototype vectors to explain the treatment recommendation for decision making purposes by medical personnel.

8. The method of claim 6, further comprising automatically implementing the treatment recommendation at a client system to treat the patient.

9. The method of claim 1, wherein the parameterized vectors include skill prototypes relating to expert demonstrations in an imitation learning system.

10. The method of claim 1, further iteratively receiving updated locally trained parameters and distributing the updated global parameters and the updated local prototype vectors.

11. A system for skill prediction, comprising:
a hardware processor; and
a memory that stores a computer program which, when executed by the hardware processor, causes the hardware processor to:
aggregate locally trained parameters from a plurality of client systems to generate updated global parameters;
cluster parameterized vectors from the client systems into prototype clusters;
determine a centroid of each prototype cluster;
match the parameterized vectors from the client systems to centroids of the prototype cluster to identify sets of updated local prototype vectors; and
distribute the updated global parameters and the updated local prototype vectors to the plurality of client systems.

12. The system of claim 11, wherein the computer program further causes the hardware processor to average the locally trained parameters.

13. The system of claim 11, wherein the computer program further causes the hardware processor to identify a mean of vectors in the prototype cluster.

14. The system of claim 11, wherein the locally trained parameters include parameters of a convolution layer and an imitation learning layer of a skill prediction model.

15. The system of claim 14, wherein the skill prediction model is implemented as a machine learning model.

16. The system of claim 14, wherein the skill prediction model accepts a patient's state as input and outputs a treatment recommendation.

17. The system of claim 16, wherein the treatment recommendation includes an interpretable identification of local prototype vectors to explain the treatment recommendation for decision making purposes by medical personnel.

18. The system of claim 16, wherein the computer program further causes the hardware processor to automatically implement the treatment recommendation at a client system to treat the patient.

19. The system of claim 11, wherein the parameterized vectors include skill prototypes relating to expert demonstrations in an imitation learning system.

20. The system of claim 11, wherein the computer program further causes the hardware processor to iteratively receive updated locally trained parameters and distributing the updated global parameters and the updated local prototype vectors.
